(19) United States
(12) Patent Application Publication
(10) Pub. No.: US 2008/0125796 A1
(43) Pub. Date: May 29, 2008

(54) Gastrotomy closure device

(75) Inventor: Marc Graham, Somerville, MA (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Stryker Development LLC, Cambridge, MA (US)

(21) Appl. No.: 11/998,345

(22) Filed: Nov. 28, 2007

Related U.S. Application Data
(60) Provisional application No. 60/861,371, filed on Nov. 28, 2006.
(60) Provisional application No. 60/925,533, filed on Apr. 19, 2007.

(51) Int. Cl. A61B 17/10; A61B 1/018; A61M 29/02

(52) U.S. Cl. 606/142; 600/153; 606/192

(57) ABSTRACT

Apparatus and methods for sealing an aperture in body tissue that extends from an outside surface to an inside surface of the body tissue is described. One embodiment of the apparatus includes an expandable material positioned against at least a portion of an expandable member, the expandable material having a sealing surface adapted to expand against the inside surface of the body tissue surrounding the aperture as the expandable member expands. In an alternative embodiment, tissue is pulled by a retractor mechanism between two biased legs of a deformable clip that is loaded on a trigger. As a base of the trigger is moved away from a base of a housing, the clip is released from the trigger causing the two biased legs of the clip to come together and seal tissue therebetween. Generally, the apparatus of each embodiment is delivered to an aperture site through an endoscope.

### CROSS-REFERENCE TO RELATED APPLICATIONS

This application claims the benefit of the filing date of U.S. Provisional Patent Application Nos. 60/861,371, filed Nov. 28, 2006 and 60/925,533, filed Apr. 19, 2007, the disclosures of which are hereby incorporated herein by reference.

### FIELD OF THE INVENTION

The present invention generally relates to a closure device which effectively seals an opening, surgically made or not, in an organ or elsewhere, and more specifically to the use of a bioadhesive, a bioabsorbable, or a bioresorbable material and a friction element for sealing apertures in the lining of the stomach.

### BACKGROUND OF THE INVENTION

Gastrotomy refers to a surgical opening in the stomach. During a gastrostomy procedure, an artificial external opening into the stomach may be created for nutritional support, gastrointestinal compression, or other reasons. This type of procedure typically includes an incision in the patient's epigastrium as part of a formal operation. Generally, a gastrostomy may be performed with a surgical approach or through a procedure called percutaneous endoscopic gastrostomy (“PEG”), which occurs through the abdominal wall.

A feeding tube is a medical device used to provide nutrition to patients who cannot or refuse to obtain nutrition by swallowing. The state of being fed by a feeding tube is called enteral feeding or tube feeding. In the case of chronic disabilities, a PEG procedure is performed in order to place a gastric feeding tube as a long-term means for providing nutrition to patients who cannot take food orally. Alternatively, placement of the feeding tube may be temporary for the treatment of acute conditions. In any event, many patients treated using a feeding tube lack the ability to survive on their own without such technology.

There are many types of medical conditions where placing a feeding tube into the stomach of a patient would aid the patient in either recovery or even survival. For example, many stroke patients are at risk of aspiration pneumonia due to poor control over the swallowing muscles. Some stroke patients will benefit from a PEG performed to maintain nutrition. Another medical condition in which a gastric feeding tube would aid a patient is in cases of gastric volvulus. With this condition, a PEG may be inserted to decompress the stomach.

In a gastrostomy procedure, a feeding tube may enter the body through any of a human's orifices or may enter the body percutaneously. For example, a feeding tube may be inserted through an orifice in the human body and placed endoscopically. Generally, the patient is sedated, and an endoscope is passed through the mouth and esophagus into the stomach. Endoscopy is a minimally invasive diagnostic medical procedure used to evaluate the interior surfaces of an organ by inserting a small tube into the body. Often, but not necessarily, an endoscope enters the body through a natural body opening. Through the scope one is able to see lesions and other surface conditions. Additionally, an endoscope may also be used to provide instrumentation to a surgical site.

Laparoscopic surgery is another minimally invasive surgical procedure. Medically, laparoscopic surgery refers to operations within the abdomen or pelvic cavity. Laparoscopic surgery belongs to the field of endoscopy. During a laparoscopic procedure, small incisions of up to half an inch may be made in the body and plastic tubes called ports are placed through these incisions. A camera and medical instruments may then be introduced through the ports which allow access to the inside of the patient. The camera transmits an image of the organs inside the abdomen onto a television monitor. A surgeon generally cannot see directly into the patient without a traditional large percutaneous incision. Therefore, the video camera becomes a surgeon's eyes in laparoscopic surgery, since the surgeon uses the image from the video camera positioned inside the patient's body to perform the procedure.

Once a surgeon has access to the abdominal organs in a laparoscopic or endoscopic procedure, an incision in the lining of the stomach may be made to place a feeding tube. When the gastrostomy is completed and the feeding tube is removed from the surgical site, a gastrotomy is left in the lining of the stomach. Currently, there exists a need for a proven method of effectively sealing this aperture so as to avoid leakage. The present closure method employs laparoscopic and/or endoscopic procedures to do so.

Known techniques of closing apertures within the body include, for example, stapling, stitching, and cauterizing. Each method has advantages and disadvantages with respect to the amount of time needed to seal an aperture, the quality of the seal that is created, and the benefit to the patient. While each method may be performed in a minimally invasive laparoscopic or endoscopic procedure, there exists a need for a gastrotomy closure device that creates a fast, quality seal, with long term benefits to the patient with or without using the above mentioned known techniques of sealing apertures.

While there are important considerations in choosing a method to close and seal an aperture in the lining of the stomach, quality of seal is one of the most important. Generally, a leak rate that is more than 1% is an unacceptable result. It is vital that fluids not be exchanged between the inside of the stomach and outside organs. Importantly, acids that are secreted in the stomach to aid in digestion must not leak outside of the stomach. Any leaking could cause serious post-operative complications, and in some case, even result in a patient's death.

### SUMMARY OF THE INVENTION

After a gastrostomy is performed, the present invention can be employed to seal an aperture left in the lining of the stomach. While discussing the invention in the context of a gastrostomy, the present invention is also applicable to seal other apertures in the tissues of the body.

There are many functional requirements for sealing an aperture created in the stomach following a gastrostomy procedure. Some of the functional requirements may include positioning an endoscope around an aperture created in the lining of the stomach, holding the endoscope over the aperture site, delivering surgical instruments and/or a closure device though the endoscope to the aperture site, retracting the surgical instruments and/or a portion of the closure device into the endoscope, and sealing the aperture with the closure device. Generally, sealing the aperture in the lining of the stomach is considered the most important functional requirement in a gastrostomy procedure.

In gastrostomy procedures performed laparoscopically and/or endoscopically, there is minimal access for a surgeon to seal the aperture site as compared with alternative procedures that create traditional large percutaneous incisions. In addition, a surgeon needs varying degrees of access at the aperture site to perform sealing methods such as stitching, stapling, or cauterizing. For instance, while stitching may create a quality seal, this method is time consuming and cumbersome for a surgeon to perform laparoscopically or endoscopically.

It would be beneficial to a surgeon if an aperture in tissue was initially repaired by pinching together the tissue surrounding the aperture with a bioadhesive, bioabsorbable, or bioresorbable material before the use of traditional stitching, stapling, or cauterizing methods of sealing apertures in the tissues of the body. Further, other sealing techniques are contemplated in accordance with the present invention, including the sealing of an aperture through the use of a bioadhesive, bioabsorbable, or bioresorbable material, a frictional element, or a combination of a bioadhesive, bioabsorbable, or bioresorbable material and frictional element, or the use of expedients with other expedients.

In a first embodiment of the gastrotomy or other closure device of the present invention, an endoscope may be positioned against or in the vicinity of the outside surface of a patient's stomach surrounding an aperture. Preferably, a first and second expandable material may then be delivered through the endoscope, and then through the aperture and into the inside of the stomach.

The outside or inside surface of the stomach may be referred to as stomach tissue or the lining of the stomach throughout the present application. The inside of the stomach generally refers to the volume of space encapsulated by the surrounding stomach tissue.

The first expandable material may act as a pressure element in the gastrotomy closure device. A pressure element of the present invention may be used to impart a force on a second expandable material, resulting in the second expandable material expanding and pressing against an inside surface of the stomach surrounding the aperture. Preferably, an initial seal is created between an outside surface of the second expandable material and the tissue surrounding the aperture on the inside surface of the stomach.

In the present invention, the second expandable material may be a bioadhesive, bioabsorbable, or bioresorbable material and is preferably a bioadhesive material. Preferably, the second expandable material is a thin blanket-like material having a density sufficient to inhibit the passage of fluids therethrough. A bioadhesive, bioabsorbable, or bioresorbable material is preferably a biocompatible material which, when placed against the surface of tissue, will adhere to the tissue and create a sealing bond between the material and the tissue it comes in contact with. This sealing bond may be a chemical bond from a reaction between the second expandable material and the tissue. Further, this sealing bond may be created through the adhesive properties of the second expandable material. Further still, this sealing bond may be created through the combination of a chemical bond and the adhesive properties of the second expandable material.

Additionally, the second expandable material may contain a therapeutic agent to aid in the healing process of the aperture once adhered to the tissue surrounding the aperture. The therapeutic agent may be an antibiotic agent, antimicrobial agent, anesthetic, or any combination of these. Additionally, artificial or natural tissue growth substances or compounds may also be used to promote healing, alone or in combination with the therapeutic agents. For instance, a tissue growth hormone may be delivered together with or separately from the second expandable material or other portions of the expandable materials. In one embodiment, the therapeutic agent contained within the second expandable material may be time released.

The first expandable material may be used to impart a force along the inside surface of the second expandable material. The first expandable material may be, for example, an inflatable balloon. Generally, the second expandable material includes an inside surface and an outside surface. Preferably, the inside surface of the second expandable material is initially in contact with the outside surface of the first expandable material as the first and second expandable materials enter the inside of the stomach through the aperture.

After entering the inside of the stomach, the first expandable material preferably expands. As the first expandable material expands, the second expandable material preferably expands proportionally. Eventually, the second expandable material preferably expands enough so that the outside surface of the second expandable material will press against the inside surface of the stomach surrounding the aperture. As the second expandable material continues to expand, friction is generated between the outside surface of the second expandable material and the inside surface of the stomach surrounding the aperture. This frictional force may act as activation energy for a reaction between the second expandable material and the stomach tissue it comes in contact with. A combination of friction forces and adhesive properties of the second expandable material may act together to create an initial seal surrounding the tissue on the inside surface of the aperture. Preferably, the second expandable material acts as a patch to facilitate a seal of the aperture.

In one embodiment, after the second expandable material and the inside lining of the stomach surrounding the aperture have initially bonded, the first expandable material may reduce in area or deflate. When the first expandable material has sufficiently reduced in area, it can be removed from the inside of the second expandable material through an opening preferably in the second expandable material. The first expandable material may be pulled back out through the aperture in the lining of the stomach and then back out through the endoscope from which it entered into the stomach.

As the first expandable material is removed through the opening in the second expandable material, the opening may seal on its own due to contracting forces created around the circumference of the opening as the opening is stretched to allow passage of the first expandable material therethrough. Further, the adhesive properties of the second expandable material may aid in the natural sealing of the opening in the second expandable material. Preferably, in one embodiment, the combination of contracting forces and the adhesive properties of the second expandable material allow the opening in the second expandable material to seal effectively on its own. In each case, the aperture is effectively sealed to prohibit the exchange of materials through the aperture between the inside of the stomach and the surrounding outside organs.

In one embodiment, after the first expandable material has been removed from the inside of the second expandable material, preferably a portion of an at least one retaining mechanism extending outwardly from the outside surface of the second expandable material is located above the outside surface of the stomach. A point is defined as the location in which the at least one retaining mechanism extends outwardly from the outside surface of the second expandable material. This point is preferably adjacent to a line along the plane of the outside surface of the stomach.

Preferably, a retractor has a first end configured to engage the at least one retaining mechanism for pulling a portion of the outside surface of the second expandable material outside of the aperture. Preferably, this step may be performed in order to ensure that the second expandable material is tightly sealed to the tissue surrounding the aperture. Additionally, the retractor may also aid in further sealing the opening in the second expandable material where the first expandable material is removed from.

After the second expandable material has created a seal around the circumference or perimeter of the gastrotomy an opening may still be left in the second expandable material after the first expandable material is removed. In most cases, the second expandable material may self seal due to the physical properties of the material. If the second expandable material does not self seal, the retractor may be used to engage the at least one retaining mechanism of the second expandable material to seal the second expandable material by forcing portions of the material surrounding the opening in the second expandable material together.

While the first expandable material may have created an initial bond between the second expandable material and the inside surface of the lining of the stomach through frictional forces and the adhesive properties of the second expandable material, the retractor pulling a portion of the second expandable material outside of the aperture may be used to ensure a continuous seal of the second expandable material to the tissue surrounding the aperture.

In one embodiment, further steps may be taken in order to seal an aperture in the lining of the stomach. In this alternative embodiment, a laparoscope or an endoscope may be positioned against or in the vicinity of the outside surface of a patient's stomach surrounding an aperture. Preferably, a first expandable material and a second expandable material may then be delivered through the endoscope, and then through the aperture and into the inside of the stomach.

The first expandable material may act as a pressure element in the gastrotomy closure device. This pressure element of the present invention may be used to impart a force on a second expandable material, resulting in the second expandable material expanding and pressing against an inside surface of the stomach surrounding the aperture. Preferably, an initial seal is created between the second expandable material and the tissue surrounding the aperture on the inside surface of the stomach. A combination of friction forces and adhesive properties of the second expandable material may act together to initially seal the aperture before further steps are taken to ensure the sealing of the aperture in the lining of the stomach.

In one embodiment, the second expandable material may be expanded to adhere to the inside surface of the stomach. In a separate pinching step, a retractor may engage the retaining mechanism of the second expandable material, thereby pulling the second expandable material into the endoscope until a portion of the tissue surrounding the aperture is also pulled into the endoscope. Preferably, the tissue surrounding the aperture is now located inside the internal walls of the endoscope.

Further, a portion of the outside surface of the second expandable material may alternatively be pulled into the endoscope by vacuum pressure. As tissue is vacuum pulled into the endoscope, the surgeon may then stitch, staple, or cauterize the pinched and/or pulled tissue to seal the aperture.

A further aspect of this embodiment, includes a surgeon having easy access to seal the already pinched tissue surrounding the aperture with various suitable methods of sealing apertures, such as stitching, stapling, or cauterizing. This embodiment preferably uses the combination of an expandable material, a frictional element, and the methods of stitching, stapling, or cauterizing, or combinations of each of the sealing methods.

In a second embodiment, a second expandable material may be alternatively placed on the inside surface of a first expandable material. The first expandable material may be a spring loaded mechanism and the second expandable material may be a bioadhesive. Preferably, the second expandable material is a bioadhesive.

The first expandable material may be inserted through an endoscope and then into the inside of the stomach through an aperture in the lining of the stomach. Preferably, the first expandable material has a first and second end. The first end is the part of the first expandable material that first enters through the aperture in the lining of the stomach after traveling through the endoscope. Preferably, the first end of the first expandable material has a smaller diameter than that of the second end. Further, the second end preferably has a natural inclination to extend outwardly in a radial direction if there is no opposing force acting on the second end.

When there is no opposing force on the outside surface of the second end of the first expandable material, the second end preferably extends outwardly in a radial direction. However, as the first expandable material passes through the endoscope, the internal walls of the endoscope create the opposing force necessary to keep the first expandable material in an unexpanded position. After the first expandable material has been fully inserted through the endoscope into the inside of the stomach, the second end of the first expandable material extends outwardly. Preferably, the expanded diameter of the second end substantially prohibits the first expandable material from retracting back through the aperture of the stomach.

After the second end of the first expandable material has extended outwardly or expanded, the second expandable material located on the inside surface of the first expandable material has expanded proportionally. The first expandable material may then be pulled in a direction opposed to that of which it entered into the aperture. As this occurs, the first expandable material becomes inverted causing the outside surface of the bioadhesive second expandable material to now be pressed against the inside surface of the tissue surrounding the aperture. Preferably, an initial seal is created between the outside surface of the second expandable material and the inside surface of the tissue surrounding the aperture.

Preferably, the first expandable material is then pulled further into the endoscope causing a portion of the tissue sealed to the second expandable material surrounding the aperture to pinch together and be pulled inside the endoscope to ensure the aperture is continuously sealed.

In one embodiment, the first expandable material would remain partially inside and partially outside of the stomach as the aperture is sealed.

In one embodiment, the second expandable material initially bonds to the inside surface of the tissue surrounding the aperture.

The first expandable material may then be pulled in a direction opposed to that of which it entered into the aperture. As this occurs, the first expandable material becomes inverted. As the first expandable material begins to pass through the endoscope in an inverted state, the outside surface of the second expandable material is sealed to the inside surface of the tissue surrounding the aperture and the outside surface of the tissue surrounding the aperture is now pressed against the internal walls of the endoscope. At this time, the second expandable material is pinching the tissue surrounding the aperture and therefore causing the tissue surrounding the aperture to be pulled inside the endoscope. Further, a portion of the outside surface of the second expandable material may also be pulled into the endoscope by vacuum pressure. As tissue is vacuum pulled into the endoscope, the surgeon may then stitch, staple, or cauterize the tissue to seal the aperture.

A separate stitching, stapling, cauterizing or other closing or sealing step may be performed on the pinched tissue to ensure the sealing of the aperture. In one embodiment, a surgeon will now have easy access to seal the already pinched tissue surrounding the aperture with known techniques of sealing apertures, such as stitching, stapling, or cauterizing.

In one embodiment of the invention, an apparatus for closing an aperture in the tissue of an organ includes a first expandable material positioned against an inside surface of a second expandable material. The second expandable material may be a bioadhesive, a bioabsorbable, or a bioresorbable material having an outside surface configured to expand along the inside surface of the stomach as the first expandable material is expanded. At least one retaining mechanism may extend outwardly from the outside surface of the second expandable material. A retractor may have a first end configured to engage the at least one retaining mechanism for pulling a portion of the second expandable material outside of the aperture.

In one aspect of the embodiment, the apparatus may further include an endoscope, the endoscope configured to allow passage of the first expandable material and the second expandable material therethrough in order for the first expandable material and the second expandable material to enter the inside the stomach through the aperture in the lining of the stomach.

In addition, the first expandable material may be an inflatable balloon.

In addition, the second expandable material may further include a therapeutic agent.

In addition, the first end of the retractor may be configured as a hook. The first end may engage the at least one retaining mechanism of the second expandable material by hooking, pinching, or gripping the at least one retaining mechanism.

In addition, the second expandable material may include an opening configured to allow the second expandable material to exit therefrom.

In another aspect of the invention, an apparatus for closing an aperture in the lining of the stomach includes a first expandable material having a second expandable material located on the inside surface of the first expandable material, the second expandable material having an inside surface configured to expand along the inside surface of the stomach as the first expandable material is expanded, at least one retaining mechanism extending outwardly from the outside surface of the second expandable material and a retractor having a first end configured to engage the at least one retaining mechanism for pulling a portion of the second expandable material outside of the aperture.

A method of the present invention for sealing an aperture in the lining of the stomach includes placing a first end of an endoscope or laparoscope against or in the vicinity of the outside surface of the stomach surrounding an aperture, delivering both a first expandable material and a second expandable material into the inside of the stomach through the endoscope and through the aperture, the second expandable material having at least one retaining mechanism extending outwardly from an outside surface, expanding the first expandable material against an inside surface of the second expandable material, thereby causing the second expandable material to expand along an inside surface of the stomach surrounding the aperture, reducing the first expandable material, removing the first expandable material from the aperture, engaging the at least one retaining mechanism of the second expandable material with a retractor, and pulling a portion of the second expandable material outside the aperture in the lining of the stomach, thereby ensuring a continuous seal around the tissue surrounding the aperture.

In yet another aspect of the invention, an apparatus for closing an aperture in the lining of the stomach or another organ or portion of the body preferably includes a trigger, a housing and a clip. The trigger may include a base having two legs extending substantially perpendicular therefrom, the two legs being slidably connected to the housing. The housing may include a proximal end and a distal end. The clip may include a base having two biased legs which are normally “closed” by a spring force or any suitable means. The term closed with respect to the clip refers to the legs touching or having a portion of tissue pinched therebetween. The biased legs preferably have hooked ends extending therefrom, wherein the hooked ends engage the legs of the housing when in an “open” position.

In a first position, the base of the trigger may, in one aspect of the invention be adjacent to the proximal end of the housing while the base of the clip may be adjacent to the distal end of the housing. The base of the clip may be releasably retained at the distal end of the housing, the housing configured to prevent the clip from moving towards the proximal end of the housing. In the first position, the two biased legs of the clip preferably extend substantially perpendicular from the base of the clip. In the first position, the hooked ends of the two biased legs preferably releasably retained on the two legs of the trigger. In a second position, the base of the trigger may be positioned further away from the distal end of the housing. In the second position, the hooked ends of the two biased legs may be released from the two legs of the trigger. The two biased legs may be released from the two legs of the trigger as the base of the trigger is moved further away from the distal end of the housing than it was when in the first position. Preferably, the base remains in a fixed position while the trigger and the clip move with respect to the base. Upon the two biased legs of the clip being released from the leg of the trigger, the two biased legs may thereby move together and seal pinched tissue therebetween.

In another aspect of the invention, the trigger, the housing, and the clip may all have a central axis. When the trigger, the housing, and the clip are initially assembled, the central axes of the trigger, the housing, and the clip are preferably substantially aligned.

In another aspect of the invention, the housing may be c-shaped wherein the housing has a base having a proximal end and a distal end and two legs extending substantially perpendicular from the distal end of the housing. The c-shaped housing allows more tissue to be pulled into the loaded clip without having the tissue interfered with by the distal end of the housing.

In another aspect of the invention, the trigger, the housing, and the clip may all have an opening that is formed substantially coaxial with their central axes. The openings in the trigger, the housing, and the clip are configured to allow for surgical instruments and/or a retracting mechanism to pass therethrough.

In another aspect of the invention, an apparatus for closing an aperture in the lining of the stomach or other organ or portion of the body may include a central shaft having a central axis and an opening coaxial with the central axis of the central shaft. The outside perimeter of the central shaft is preferably configured to fit and move transversely in a forward and backward direction through the openings of the trigger, housing, and a clip that can be closed to retain tissue surrounding an aperture when in a closed position. The central shaft is preferably configured to contain hardware used for grabbing tissue and pulling it into the clip loaded onto the two legs of the trigger. Non-exhaustive examples of such hardware are a retractor, a bioadhesive, and a balloon as described in the embodiments above. It is within the scope of the invention to include other hardware known in the art such as for example hooks, clamps, and clips.

In another aspect of the invention, the central shaft may include an engaging means. The engaging means may be, for example, a notch located in the outside surface of the central shaft. The engaging means may alternatively be, for example, a protrusion on the outside surface of the central shaft. The non-exhaustive examples of a notch and protrusion will preferably be configured to engage a corresponding or mating structure of the trigger. For example, the central shaft may have a male feature and the trigger may have a female feature that mate or vice versa.

After the central shaft has been delivered to the aperture site through an endoscope for example, and the hardware has been deployed to pull tissue between the loaded clip, the central shaft is then pulled in the opposite direction back through the endoscope. As the central shaft is pulled further back into the endoscope, the engaging means of the central shaft eventually engages a mating feature on the trigger thereby pulling the trigger in the same direction as that of the central shaft. The two biased legs of the clip are preferably released from the two legs of the trigger as the base of the trigger is moved further away from the distal end of the housing. As this occurs, the base of the clip is prevented from moving towards the proximal end of the housing thereby causing the two biased legs of the clip to be released from the two legs of the trigger and move together and seal tissue therebetween.

In yet another aspect of the invention, an apparatus for closing an aperture in the lining of the stomach or another organ or portion of the body preferably includes an endoscope that engages the distal end of the housing and cannot pass therethrough to the proximal end of the housing. The endoscope may be provided to act as a passageway for hardware to pass therethrough.

In addition, the apparatus may include an overtube containing a first bar and a second bar. The first and second bar may be connected to the base of the trigger for pulling the base of the trigger further away from the distal end of the housing and releasing the clip. Alternatively, the first and second bar may be attached to the housing and provide a track means for the trigger slidably connected to the housing to move further away from the distal end of the housing as the endoscope engages the trigger. The overtube may be attached to and separated from the housing a fixed distance. The overtube is preferably configured to guide the trigger, housing, clip, and endoscope to the aperture site in tissue. The overtube may be configured to provide a passageway for hardware delivered to the aperture site.

In addition, the first and second bars may be, for example, wires, cables, or rods. The first and second bars may be attached to the trigger. The first and second bars may extend out of the overtube and may be accessible for a surgeon to pull further outside of the overtube. Preferably, as the first and second bars are pulled, the trigger is pulled away from the distal end of the housing and the two biased legs of the clip are released from the two legs of the trigger. The two biased legs may thereby move together and seal tissue therebetween.

In addition, an additional endoscope may contain the overtube, the first and second bars, the endoscope, the trigger, the housing, and the clip. The additional endoscope may be positioned around an aperture in the lining of the stomach or another organ or portion of the body. Preferably, the distal end of the additional endoscope, the end that surrounds the aperture, is c-shaped. This shape preferably allows more tissue to be placed between the biased legs of the clip in the open position before the clip is released to seal the tissue at the aperture site.

Preferably, the overtube, the first and second bars, the endoscope, the trigger, the housing, and the clip may all be contained within the additional endoscope as the additional endoscope is positioned around the aperture. Preferably, the additional endoscope is held over the aperture site while surgical instruments, hardware and/or a closure device are delivered though the endoscope to the aperture site. A combination of a retractor mechanism, bioadhesive, and/or balloon may be delivered into the aperture through the lining of the stomach or another organ or portion of the body and then used to retract at least a portion of tissue into the endoscope containing the closure mechanism. The retracted tissue preferably is pulled between the biased legs of the clip of the closure mechanism when the clip is in the open position. The trigger holds the clip in the open or loaded position. Preferably, when the trigger is moved further away from the distal end of the housing by the first and second bars attached to the trigger, the trigger releases the clip. Preferably, the clip, when released by the trigger, closes down on the retracted tissue to seal the gastrotomy. After the clip has been released, all instruments may be removed from the aperture site. Preferably, the additional endoscope containing the overtube, first and second bars, endoscope, trigger, housing, and clip may all be removed.

An alternative method of the present invention for sealing an aperture in the lining of the stomach or another organ or portion of the body may include placing a first end of an endoscope or laparoscope against or in the vicinity of the outside surface of the stomach surrounding an aperture. The endoscope containing a retractor mechanism and a closure device. The retractor mechanism including a cable having an engaging means, for example, at least one hooked member at the distal end of the engaging means for engaging tissue surrounding the aperture. The closure device including the trigger, the housing, and the clip. The closure device may also include the elements of the retractor mechanism.

The method may further include delivering the retractor mechanism through the aperture and into the lining of the stomach or another organ or portion of the body and then deploying the engaging means of the retractor mechanism. The retractor mechanism may then be pulled back through the aperture in the lining of the stomach or another organ or portion of the body so that the engaging means may engage the tissue surrounding the aperture and pull the tissue between the biased legs of the clip, the clip being releasably retained on the legs of the trigger. As the retractor continues to be pulled back through the endoscope which it originally passed through, the trigger of the closure mechanism is pulled further away from the distal end of the housing thereby releasing the clip onto the pulled back tissue. The clip is thereby released from the trigger. The retractor mechanism, the trigger and the housing may now be removed with the endoscope from the aperture site.

In yet another aspect of the invention, an apparatus for closing an aperture in the lining of the stomach or another organ or portion of the body preferably comprises an overtube fixedly attached to a housing, the housing including a proximal end, a distal end, a central axis and an opening. The opening of the housing preferably extends from the proximal end through the distal end, the housing having a substantially fixed position once the closure device is delivered to the aperture site. The apparatus may further include a trigger slidably mounted on the housing, the trigger including a base, two legs arranged substantially perpendicular to the base, a central axis and an opening. The central axis of the trigger is preferably aligned with the central axis of the housing. The trigger preferably has a first position and a second position, wherein the base of the trigger is adjacent to the proximal end of the housing in the first position, and the base of the trigger is further away from the distal end of the housing in the second position than it is in the first position.

The apparatus may further include a clip including a base, two biased legs, a central axis and an opening, the clip having a first position and a second position. In the first position the base of the clip is adjacent to the distal end of the housing and each of the two legs of the clip is engaged by one of the two legs of the trigger. The apparatus may further include a central shaft including at least one engagement portion and a central axis, the central axis of the central shaft aligned with the central axis of the housing. The central shaft may be configured to be received within the opening of the housing and translate in both a forward first direction and a second backward direction along the central axis of the housing. Preferably, the at least one engagement portion of the central shaft engages the base of the trigger during movement of the central shaft in the second direction thereby releasing the two biased legs of the clip from engagement with the two legs of the trigger. The second position of the clip may be defined as the two biased legs of the clip touching or having pinched tissue therebetween thereby sealing the tissue.

### BRIEF DESCRIPTION OF THE DRAWINGS

FIG. 1 is a general view of a human stomach having an aperture through the lining of the stomach.

FIG. 2 is a view of a gastrotomy closure device of the present invention just before it enters through the aperture and into the lining of the stomach.

FIG. 3 is a view of the gastrotomy closure device of FIG. 2, as the device enters the through the aperture and into the lining of the stomach.

FIG. 4 is a view of the gastrotomy closure device of FIG. 2, after the device has entered the through the aperture and into the lining of the stomach or another organ or portion of the body, wherein an at least one retaining mechanism extends outwardly from the outside surface of a second expandable material, the at least one retaining mechanism remaining outside of the lining of the stomach.

FIG. 5 is a view of the gastrotomy closure device of FIG. 2, showing a first expandable material expanded against the inside surface of the second expandable material causing the second expandable material to expand and begin to adhere along the inside surface of the stomach surrounding the aperture.

FIG. 6 is a view of the gastrotomy closure device of FIG. 2, showing the first expandable material fully expanded against the inside surface of the second expandable material.

FIG. 7 is a view of the gastrotomy closure device of FIG. 2, showing the first expandable material in an unexpanded form and being removed through an opening in the second expandable material.

FIG. 8 is a view of the gastrotomy closure device of FIG. 2, showing the first expandable material being removed through an opening in the second expandable material.

FIG. 9 is a view of the gastrotomy closure device of FIG. 2, showing the first expandable material just before it is completely removed through an opening in the second expandable material.

FIG. 10 is a view of the gastrotomy closure device of FIG. 2, showing the second expandable material adhered to the inside surface of the lining of the stomach.

FIG. 11 is a view of the gastrotomy closure device of FIG. 2, showing a retractor engaging the at least one retaining mechanism of the second expandable material.

FIG. 12 is a view of the gastrotomy closure device of FIG. 2, showing the retractor pulling the at least one retaining mechanism of the second expandable material.

FIG. 13 is a view of the gastrotomy closure device of FIG. 2, showing the aperture sealed.

FIG. 14 is a view of an alternative embodiment of a gastrotomy closure device of the present invention, showing a first expandable material traveling through an endoscope before entering into the inside of the stomach through an aperture in the lining of the stomach.

FIG. 15 is a view of the gastrotomy closure device of FIG. 14, showing a second end of the first expandable material in an expanded state.

FIG. 16 is a view of the gastrotomy closure device of FIG. 14, showing the tissue surrounding the aperture being retracted inside of the endoscope.

FIG. 17 is a view of an alternative embodiment of a gastrotomy closure device of the present including a trigger, a housing, and a deformable clip.

FIG. 18 is a view of the gastrotomy closure device of FIG. 17, wherein the clip is shown released from the trigger.

FIG. 19 is a view of the gastrotomy closure device of FIG. 17, wherein the trigger and housing is removed from the sealed aperture site.

FIG. 20 is a view of the gastrotomy closure device of FIG. 17, engaged with an alternative retractor mechanism including a cable having an engaging means.

FIG. 21 is a view of the gastrotomy closure device of FIG. 17, wherein the retractor mechanism is delivered through an aperture in the lining of the stomach.

FIG. 22 is a view of the gastrotomy closure device of FIG. 17, wherein the engaging means of the retractor mechanism is shown deployed.

FIG. 23 is a view of the gastrotomy closure device of FIG. 17, wherein the engaging means is shown just before it engages the tissue surrounding the aperture.

FIG. 24 is a view of the gastrotomy closure device of FIG. 17, wherein a portion of the engaged tissue is pulled or retracted into the endoscope or in the vicinity of the distal end of the endoscope and positioned between the biased legs of the deformable clip when the clip is in the open position.

FIG. 25 is a view of the gastrotomy closure device of FIG. 17, showing the clip being released from the trigger.

FIG. 26 is a view of the gastrotomy closure device of FIG. 17, showing the retractor mechanism, the trigger and the housing being removed with the endoscope from the aperture site.

FIG. 27 is an alternative embodiment of a gastrotomy closure device including a central shaft, a trigger, a housing, and a deformable clip.

FIG. 28 is a view of the gastrotomy closure device of FIG. 27, showing the central shaft moved in a forward direction towards the aperture site.

FIG. 29 is a view of the gastrotomy closure device of FIG. 27, showing the central shaft moved in a backward direction away from the aperture site.

FIG. 30 is a view of the gastrotomy closure device of FIG. 27, showing the central shaft moved further in the backward direction away from the aperture site, wherein an engaging means of the central shaft is located between the biased legs of the clip.

FIG. 31 is a view of the gastrotomy closure device of FIG. 27, showing the central shaft moved even further in the backward direction away from the aperture site, wherein the engaging means of the central shaft is contained within the housing.

FIG. 32 is a view of the gastrotomy closure device of FIG. 27, showing the central shaft moved even further in the second direction and engaged with the trigger.

FIG. 33 is a view of the gastrotomy closure device of FIG. 27, showing the central shaft moved even further in the second direction, wherein the hooked ends of the clip have begun to separate from the legs of the trigger.

FIG. 34 is a view of the gastrotomy closure device of FIG. 27, wherein the clip has been released from the trigger.

FIG. 35 is a view of the gastrotomy closure device of FIG. 27, wherein the central shaft, the trigger, and the housing are being removed from the aperture site away from the deployed clip.

FIG. 36 is an alternative embodiment of a gastrostomy closure device of the present invention including an endoscope, a housing, a trigger, and a deformable clip.

FIG. 37 is a view of the gastrotomy closure device of FIG. 36, a base of the trigger has been pulled further away from a distal end of the housing by movement of the endoscope in the same direction.

FIG. 38 is a view of the gastrotomy closure device of FIG. 36, wherein the clip has been released from the trigger.

FIG. 39 is a view of the gastrotomy closure device of FIG. 36, wherein the endoscope, the trigger, and the housing are being removed from the aperture site away from the deployed clip.

FIG. 40 is an alternative embodiment of a gastrotomy closure device of FIG. 36, showing an overtube having first and second bars connected to the trigger of the gastrotomy closure device of FIG. 36.

FIG. 41 is a view of the gastrotomy closure device of FIG. 36, showing the clip released from the trigger.

### DETAILED DESCRIPTION

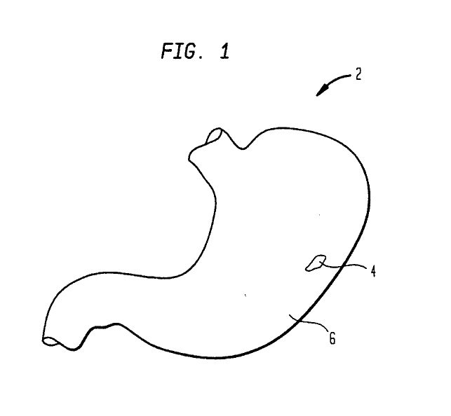

Referring to FIG. 1, a general view of a human stomach 2 having an aperture 4 extending from an outside surface 6 to an inside surface 8 of the lining of the stomach or another organ or portion of the body is shown. In a gastrostomy, aperture 4 is generally created artificially. However, the gastrotomy closure device 10 of the present invention may be used to seal any aperture in the human body, whether created surgically or naturally as the result of a trauma or disease. Preferably, the gastrotomy closure device is used for apertures in the abdominal or pelvic region; however, it should be understood that the device of the present invention may be utilized to seal apertures in any organ, including but not limited to the heart, lungs, intestines, pancreas, kidneys, and liver.

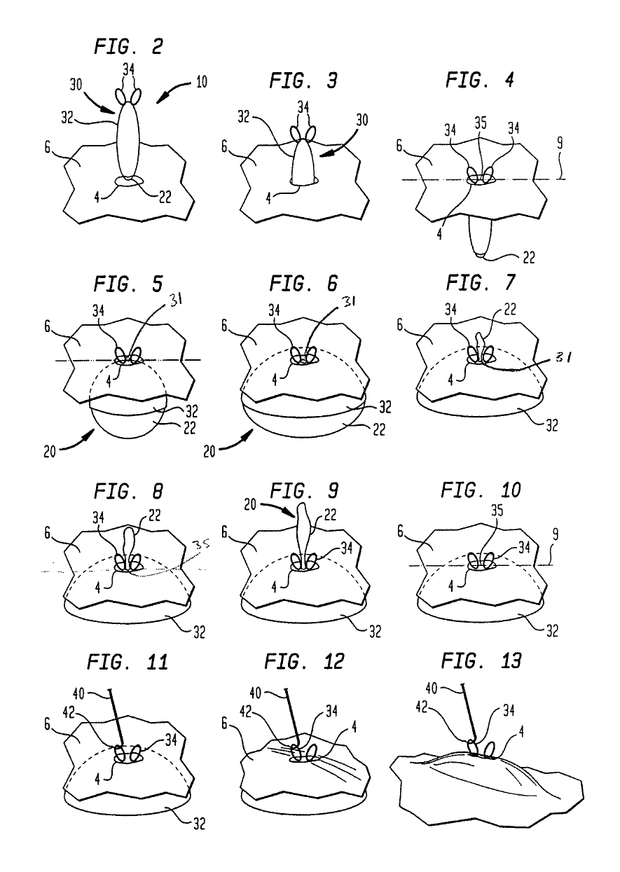

FIGS. 2-13 show a first embodiment of the gastrotomy closure device 10 of the present invention. Specifically, FIG. 2 shows a first expandable material 20 and a second expandable material 30 of gastrotomy closure device 10 just before the closure device enters through aperture 4 from the outside to the inside of stomach 2. In the present invention, first expandable material 20 may also be referred to as expandable member 20 and second expandable material 30 may also be referred to as expandable material 30. At this time, first expandable material 20 may be enclosed within second expandable material 30. Preferably, second expandable material 30 is positioned against at least a portion of first expandable material 20, wherein an outside surface 22 of first expandable material 20 is pressed against an inside surface 33 (inside surface of second expandable material as shown in FIG. 16 of alternative embodiment) of second expandable material 30. Preferably, first expandable material or expandable member 20 may be an inflatable balloon and second expandable material or expandable material 30 may be a bioadhesive, bioabsorbable, or bioresorbable material for providing a sealing function.

FIG. 3 is a view of gastrotomy closure device 10 entering into aperture 4 of stomach 2. Here, a portion of closure device 10 is shown above outside surface 6 of stomach 2 and below inside surface 8 of stomach 2.

FIG. 4 is a view of gastrotomy closure device 10 after the device has fully delivered through aperture 4 and into stomach 2. Preferably, a point 35 where an at least one retaining mechanism 34 extends outwardly from second expandable material 30 is adjacent to a line 9 along the plane of top surface 6 of stomach 2. Point 35 does not have to be exactly along line 9. Preferably, point 9 is about ±25 mm in the vertical direction from line 9. More preferably, point 9 is about ±10 mm in the vertical direction from line 9. Preferably, point 35 stays in the positive vertical direction of line 9 so that the entire at least one retaining mechanism 34 remains above outside surface 6 of stomach 2 wherein a first end 42 of a retractor 40 can easily engage at least one retaining mechanism 34.

FIG. 5 is an expanded view of gastrotomy closure device 10. Outside surface 22 of first expandable material 20 is shown in a semi-expanded state. In the present invention, semi-expanded refers to the fact that first expandable material 20 may still be further expanded. The expansion of outside surface 22 of first expandable material 20 can be seen having a proportional expanding effect on second expandable material 30. Specifically, outside surface 22 is pressed against inside surface 33 (inside surface of second expandable material as shown in FIG. 16 of alternative embodiment) of second expandable material 30. As expandable member 20 continues to expand, second expandable material 30 begins to adhere along inside surface 8 of stomach 2. At this time, first expandable material 20 may still need to expand further in order for second expandable material 30 to create an initial seal of aperture 4.

In FIG. 6, gastrotomy closure device 10 is shown in a fully expanded state. Specifically, first expandable material 20 has expanded second expandable material 30 so that outside surface 32 has adhered along inside surface 8 of stomach 2 to tissue surrounding aperture 4. Preferably, frictional forces are generated between outside surface 32 of second expandable material 30 and inside surface 8 of stomach 2 surrounding aperture 4. These frictional forces may act as activation energy for second expandable material 30. A combination of friction forces and adhesive forces between second expandable material 30 and tissue along inside surface 8 of stomach 2 surrounding aperture 4 aid in sealing aperture 4. As second expandable material 30 is pressed against the inside surface 8 of the stomach 2 of the aperture 4, a chemical reaction may begin to take place between second expandable material 30 and inside surface 8 of stomach 2 that second expandable material 30 comes in contact with. Preferably, second expandable material 30 and the tissue of the stomach surrounding aperture 4 become tightly adhered or bonded together. In this embodiment, second expandable 30 acts as a patch to seal aperture 4.

After second expandable material 30 and inside surface 8 of stomach 2 surrounding aperture 4 have bonded, first expandable material 20 may reduce in area or deflate as shown in FIG. 7. When first expandable material 20 has sufficiently reduced in area, it can be removed from the inside of second expandable material 30 through an opening 31 in second expandable material 30 as shown in FIGS. 7-9. First expandable material 20 may be pulled back out through aperture 4 of stomach 2 and further back out through an endoscope which provided a passageway for gastrotomy closure device 10 as it entered through aperture 4 into stomach 2. In another embodiment, expandable member 20 may be left in the inside of stomach 2.

As first expandable material 20 is removed through opening 31 in second expandable material 30, opening 31 may seal on its own due to contracting forces created around the circumference of opening 31 as opening 31 is stretched to allow passage of first expandable material 20 therethrough. Further, the adhesive properties of second expandable material 30 may aid in the natural sealing of opening 31 in second expandable material 30. Preferably, in this embodiment, the combination of contracting forces and the adhesive properties of second expandable material 30 allow opening 31 in second expandable material 30 to seal on its own.

As shown in FIG. 10, point 35 is preferably adjacent to line 9 along the plane of top surface 6 of stomach 2. Point 35 does not have to be exactly along line 9. Preferably, point 9 is ±25 mm in the vertical direction from line 9. More preferably, point 9 is ±10 mm in the vertical direction from line 9. Preferably, point 35 stays in the positive vertical direction of line 9 so that at least one retaining mechanism 34 remains above outside surface 6 of stomach 2 wherein retractor 40 can easily engage at least one retaining mechanism 34 as seen in FIG. 11.

At least one retaining mechanism 34 is configured to be engaged by retractor 40. First end 42 of retractor 40 is configured to engage at least one retaining mechanism 34 for pulling a portion of second expandable material 30 outside of aperture 4 in order to further seal aperture 4, if necessary, as shown in FIGS. 11-13. Preferably, first end 42 is configured as a hook; however, any configuration which may easily engage at least one retaining mechanism 34 may be used in the present invention. Additionally, retractor 40 may also aid in further sealing opening 31 in second expandable material 30 where first expandable material 20 is removed from.

While the expansion of first expandable material 20 may have created an initial bond between second expandable material 30 and inside surface 8 of stomach 2 through frictional forces, retractor 40 pulling a portion of second expandable material 30 outside of aperture 4 ensures a sufficient seal between second expandable material 30 and the tissue along inside surface 8 of stomach 2 surrounding aperture 4, such that the seal prevents leakage of fluids from the inside to the outside of the stomach from occurring. Preferably, aperture 4 in the lining of the stomach or another organ or portion of the body should now be fully repaired.

In an alternative embodiment of the first embodiment of gastrotomy closure device 10 of the present invention, an endoscope 50 may be positioned against or in the vicinity of the outside surface of a patient's stomach surrounding an aperture 4. Preferably, a first expandable material 20 and a second expandable material 30 may be delivered through endoscope 50, and then through aperture 4 and into the inside of stomach 2.

First expandable material 20 may act as a frictional element in gastrotomy closure device 10. A frictional element of the present invention may be used to impart a force on second expandable material 30, resulting in second expandable material 30 expanding and having at least a portion thereof pressing against an inside surface 8 of stomach 2 surrounding aperture 4. Preferably, an initial seal is created between second expandable material 30 and the tissue surrounding aperture 4 on inside surface 8 of stomach 2. A combination of friction forces and adhesive properties of second expandable material 30 may act together to initially seal aperture 4 before another step may be taken to ensure the sealing of the aperture in the lining of the stomach or another organ or portion of the body.

In one embodiment, bioadhesive 30 may be expanded to adhere to inside surface 8 of stomach 2. In a separate pinching step, second expandable material 30 is pulled into endoscope 50 until a portion of the tissue surrounding aperture 4 is also pulled into endoscope 50 between internal walls 52 of endoscope 50 as shown generally in FIG. 16. In this case, a surgeon will now have easy access to seal the already pinched tissue surrounding aperture 4 with known techniques of sealing apertures, such as by stitching, stapling, cauterizing or other sealing techniques suitable within the context of the invention. This embodiment uses the combination of a bioadhesive, a frictional element, and the methods of stitching, stapling, or cauterizing, or a combination of each of the sealing methods. The sealing technique can be synchronized with moving endoscope 50 away from the pinched tissue or while the endoscope 50 is stationary and the pinched tissue is inside the internal walls 52 of the endoscope 50.

FIGS. 14-16 show a second embodiment of gastrotomy closure device 10 of the present invention generally denoted by reference numeral 10′. In this embodiment, a second expandable material 30′ may be placed on an inside surface 23′ of a first expandable material 20′. Preferably, first expandable material 20′ has a natural tendency to expand outwardly and may include a spring loaded mechanism. Second expandable material 30′ may be a bioadhesive, a bioabsorbable, or a bioresorbable material. Preferably, second expandable material 30′ may be placed on an inside surface 23′ of first expandable material 20′.

In this embodiment of gastrotomy closure device 10′, first expandable material 20′ may be inserted into aperture 4′ of stomach 2′ through an endoscope 50. First expandable material 20′ may have a first end 26 and a second end 28. Preferably, first end 26 is the part of first expandable material 20′ that first enters through aperture 4′ of stomach 2′ after traveling through endoscope 50. Generally, first end 26 of first expandable material 10′ has a smaller diameter than that of second end 28. Further, second end 28 may have a natural tendency to extend outwardly in a radial direction if there is no opposing force acting on second end 28. Further, second end 28 may extend outwardly in a radial direction due to the forces of a spring loaded mechanism.

When there is no opposing force on outside surface 22′ of second end 28 of first expandable material 20′, second end 28 will then extend outwardly in a radial direction. However, as first expandable material 20′ passes through endoscope 50, internal walls 52 of endoscope 50 create the opposing force necessary to keep first expandable material 20′ in an unexpanded position.

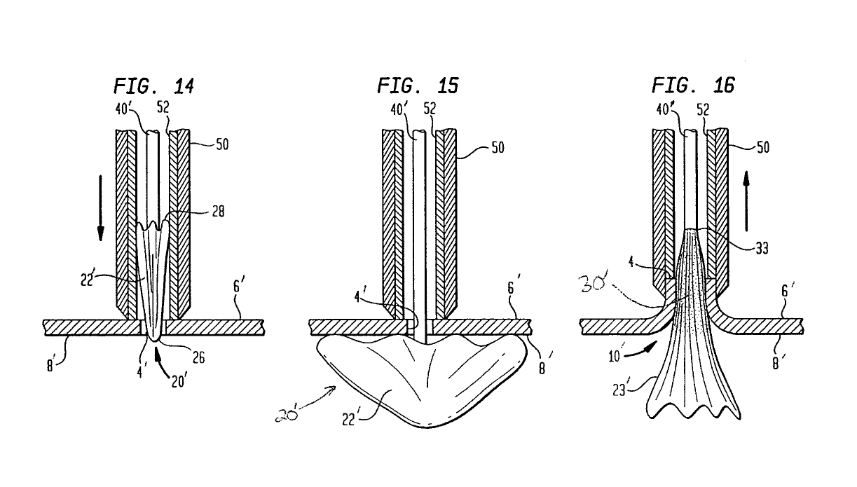

As seen in FIG. 15, first expandable material 20′ has been fully inserted through endoscope 50 and into the inside of the stomach. Second end 28 of first expandable material 20′ has extended outwardly in a radial direction. Preferably, the expanded diameter of second end 28 of first expandable material 20′ substantially prohibits first expandable material 20′ from retracting back through aperture 4′ of stomach 2′.

After second end 28 of first expandable material 20′ has extended outwardly or expanded, second expandable material 30′ located on inside surface 23′ of first expandable material 20′ preferably expands proportionally. First expandable material 20′ may then be pulled in a direction opposed to that of which it entered into aperture 4′ as depicted by the opposing arrows in FIGS. 14 and 16. As this occurs, first expandable material 20′ becomes inverted as shown in FIG. 16, causing outside surface 33 of bioadhesive second expandable material 30′ to now be pressed against inside surface 8′ of the tissue surrounding aperture 4′. Preferably, an initial seal is created between outside surface 33 of second expandable material 30′ and inside surface 8′ of the tissue surrounding aperture 4′.

Preferably, first expandable material 20′ is pulled further into endoscope 50 causing a portion of the tissue sealed to second expandable material 30′ surrounding aperture 4′ to pinch together and be pulled inside endoscope 50 to ensure aperture 4′ is continuously sealed. In this embodiment, first expandable material 20′ preferably remains partially inside and partially outside of stomach 2′ as aperture 4′ is sealed.

In one embodiment, second expandable material 30′ creates an initial bond with inside surface 8′ of the tissue surrounding aperture 4. First expandable material 20′ may then be pulled in a direction opposite that of which it entered into aperture 4′. As this occurs, first expandable material 20′ becomes inverted. As first expandable material 20′ begins to pass through endoscope 50 in an inverted state, outside surface 33 of second expandable material 30′ is now pressed against internal walls 52 of endoscope 50. Bioadhesive second expandable material 30′ may pinch the tissue surrounding aperture 4′ and therefore cause the tissue surrounding aperture 4′ to be pulled inside endoscope 50.

A separate stitching, stapling, cauterizing or other sealing step may be performed on the pinched tissue to ensure the sealing of aperture 4′. This embodiment uses the combination of a second expandable material, a frictional element, and the methods of stitching, stapling, or cauterizing, or a combination of each of the sealing methods.

An alternative embodiment of the gastrotomy closure device of the present invention is an inflatable balloon at least partially enclosed by a second expandable material. The inflatable balloon at least partially enclosed by the second expandable material enters an aperture in the stomach through an endoscope. The endoscope would be placed around an aperture in the lining of the stomach or another organ or portion of the body. A bottom portion of the endoscope would be placed along the external surface of the stomach wall.

The balloon enclosed by the second expandable material is delivered through the aperture in the lining of the stomach. After the second expandable material enters the aperture in the lining of the stomach, a retaining mechanism preferably extends outwardly from the second expandable material, the retaining mechanism remaining preferably outside of the aperture above the top surface of the stomach.

Once inside the stomach, the inflatable balloon preferably inflates and causes the stomach to expand. The second expandable material surrounding the balloon preferably expands proportionally. An outside surface of the second expandable material would eventually adhere to the inside surface of the lining of the stomach or another organ or portion of the body surrounding the aperture in the lining of the stomach.

During the time the balloon inflates friction is created between the balloon, the second expandable material and the inside surface of the stomach. When the inflatable balloon is sufficiently inflated there is preferably a tight fit or bond between the second expandable material and the inside surface of the stomach. After this bond occurs, the balloon preferably deflates. The balloon may then be pulled through an opening in the second expandable material. Once the balloon is removed a retractor including a hook mechanism may be inserted through the endoscope. The retractor may then engage at least one retaining mechanism extending outwardly from the top portion of the second expandable material.

The at least one retaining mechanism of the second expandable material may then be pulled to force a portion of the second expandable material out from the inside of the stomach. As this occurs, the circumference of the aperture is preferably pinched and an even tighter fit or seal may be created between the second expandable material and the tissue surrounding the aperture. The second expandable material now seals the aperture and becomes bonded to the surrounding tissue. Once a bond is formed, the retractor is removed from the at least one retaining mechanism of the second expandable material, and the retractor is then removed from the body back through the endoscope. The aperture in the lining of the stomach or another organ or portion of the body is preferably now repaired.

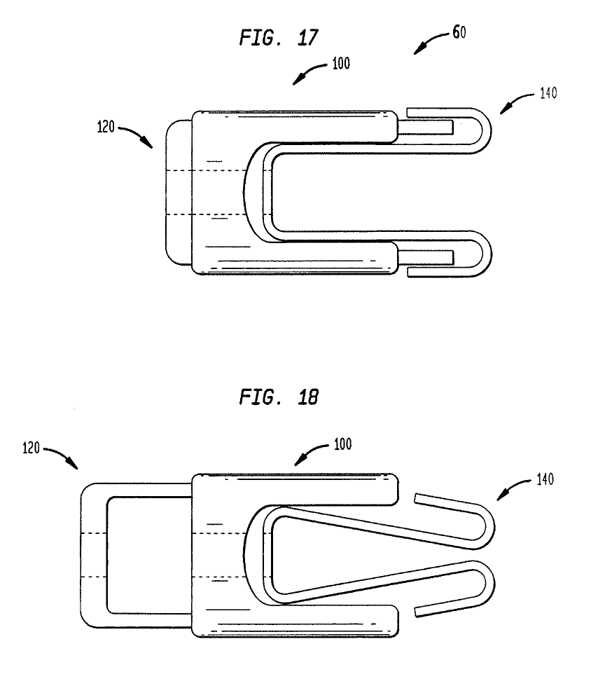

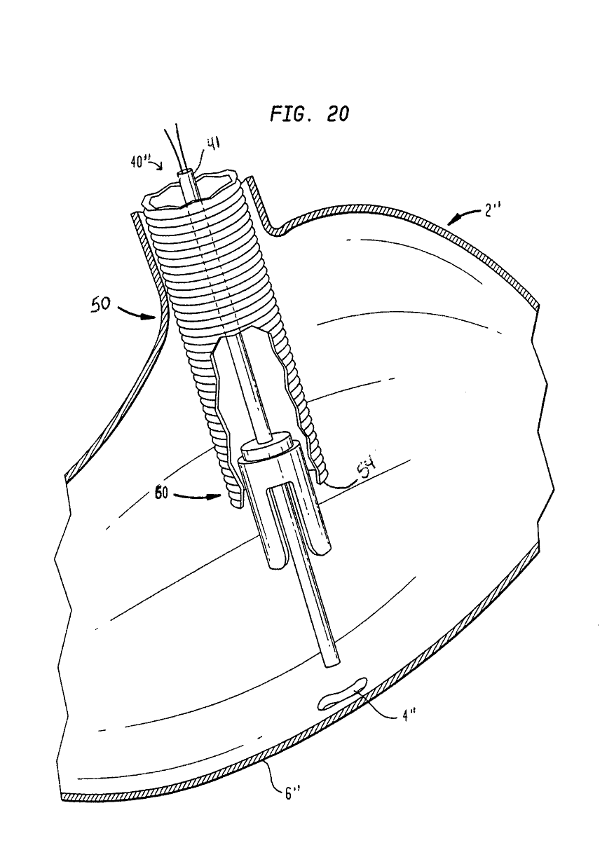

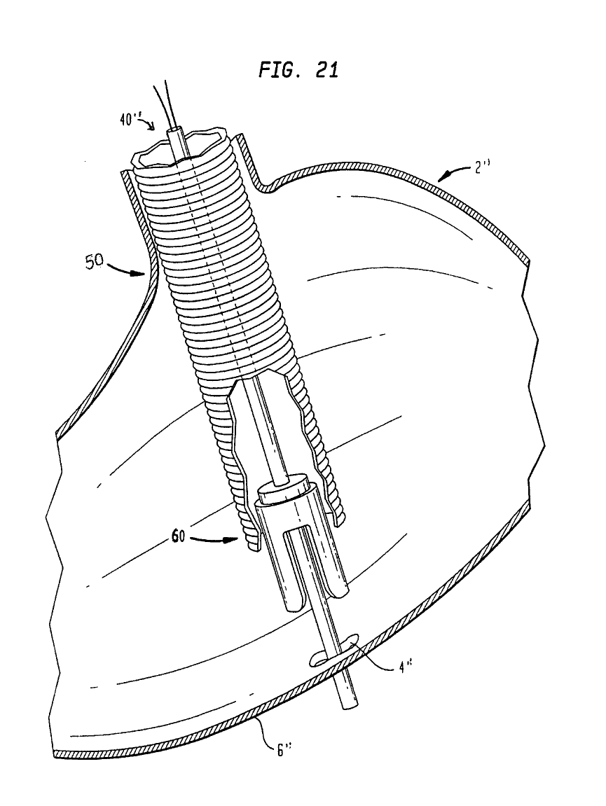

FIGS. 17-19 show an alternative embodiment of a gastrotomy closure device of the present invention. As shown in FIG. 17, a gastrotomy closure device 60 includes a housing 100, a trigger 120, and a deformable clip 140. Generally, housing 100, trigger 120, and clip 140 are initially assembled in this configuration. Gastrotomy closure device 60 is generally delivered to an aperture site through an endoscope as shown in FIG. 20.

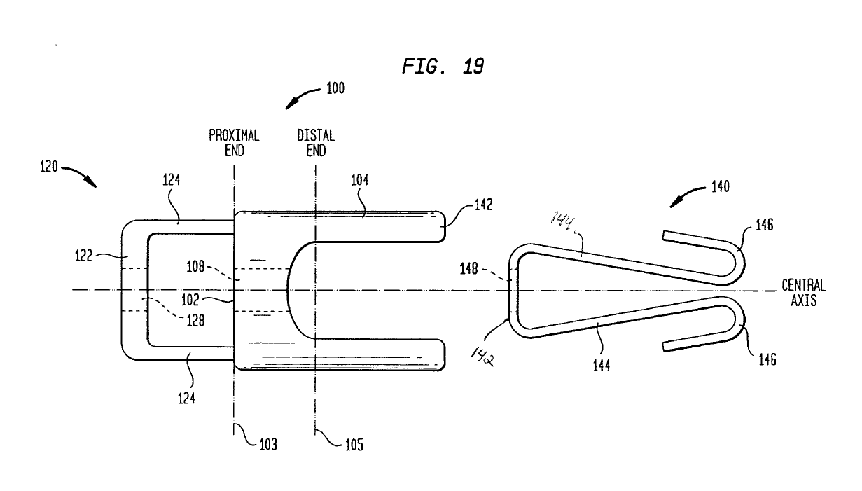

As shown in detail in FIG. 19, housing 100 includes a proximal end 103 and a distal end 105. In a preferred embodiment, housing 100 may be c-shaped wherein housing 100 has a base 102 adjacent to proximal end 103 and two legs 104 extending substantially perpendicular from the distal end 105 of the housing. The c-shaped housing allows more tissue to be pulled into clip 140 when loaded on trigger 120.

Also shown in FIGS. 17-19, trigger 120 preferably includes a base 122 having two legs 124 extending substantially perpendicular therefrom. Preferably, two legs 124 are slidably engaged to housing 100. Clip 140 preferably includes a base 142 having two biased legs 144 with hooked ends 146 extending therefrom.

Clip 140 as shown in FIG. 17 is in the “loaded” or “open” position. The term loaded or open in this context generally means that biased legs 144 of clip 140 are substantially perpendicular to base 142 of clip 140. Biased legs 144 of clip 140 have a natural tendency to move together.

In a first position as shown in FIG. 17, base 122 of trigger 120 may be adjacent to proximal end 103 of housing 100 while base 142 of clip 140 may be adjacent to distal end 105 of housing 100. Preferably, base 142 of clip 140 is releasably retained by distal end 105 of housing 100, housing 100 being configured to prevent clip 140 from moving towards proximal end 103 of housing 100. In the first position, biased legs 144 of clip 140 may extend substantially perpendicular from base 142 of clip 140. In the first position, hooked ends 146 of biased legs 144 are preferably releasably retained on legs 124 of trigger 120.

In a second position as shown in FIG. 18, base 122 of trigger 120 generally may be positioned further away from distal end 105 of housing 100 then in the first position. In the second position, hooked ends 146 of biased legs 144 are preferably released from legs 124 of trigger 120. Biased legs 144 are preferably released from legs 124 of trigger 120 as base 122 of trigger 120 is moved further away from distal end 105 of housing 100 than it was when in the first position while base 142 of clip 140 is prevented from moving towards proximal end 103 of housing 100. Biased legs 144 are thereby released from legs 124 of trigger 120 and move closer together preferably clamping tissue therebetween.

As shown in FIG. 19, trigger 120, housing 100, and clip 140 may all have a central axis, generally shown by the longitudinal dashed line. As shown in FIG. 17, when trigger 120, housing 100, and clip 140 are initially assembled, the central axes of trigger 120, housing 100, and clip 140 are preferably substantially aligned.

Trigger 120, housing 100, and clip 140 may all have an opening 128, 108, 148 respectively that is formed substantially coaxial to the central axis of trigger 120, housing 100 and clip 140. Openings 128, 108, 148 in trigger 120, housing 100, and clip 140 are configured to allow for surgical instruments and/or a retracting mechanism to pass therethrough. The retracting mechanism is preferably used to grab tissue surrounding an aperture and to pull the tissue between the biased legs 144 of clip 140 when clip 140 is in the open position.

FIGS. 20-26 show one embodiment of a method of the present invention for sealing an aperture 4″ in the lining of the stomach or another organ or portion of the body with gastrotomy closure device 60. Generally, the method includes placing a first end of an endoscope 50′ or laparoscope against or in the vicinity of an outside surface 6″ of a stomach 2″ surrounding aperture 4′.

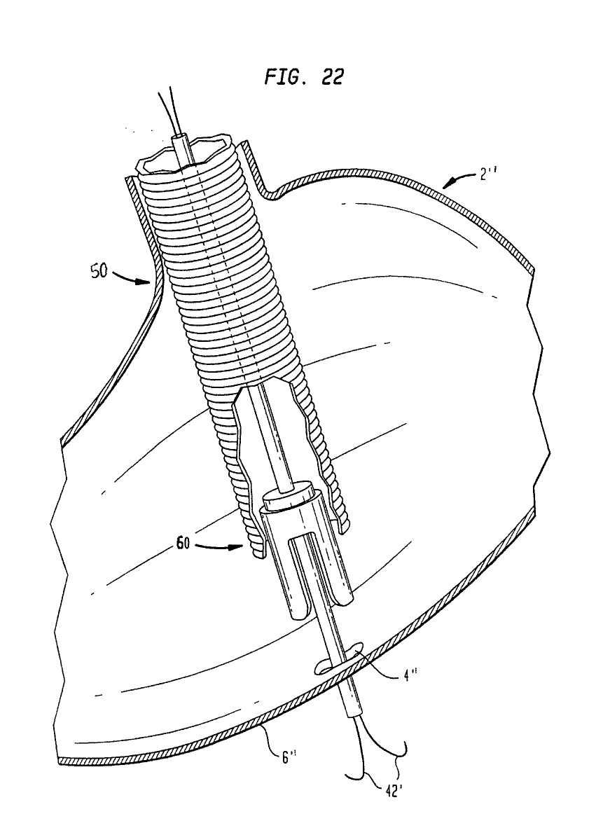

Endoscope 50′ preferably contains a retractor mechanism 40″ and closure device 60. As shown in FIGS. 20-26, closure device 60 includes trigger 120, housing 100, and clip 140 shown in FIGS. 17-19. Housing 100 of closure device 60 may be anchored at a distal end 54 of endoscope 50′ by any known positive connection, such as for example, press fit, threaded connection, or taper lock. In one embodiment, housing 100 may not be anchored to endoscope 50′. Preferably, retractor mechanism 40″ includes a cable 41 having an engaging means 42′ therein, engaging means 42′ including any known means of approximating tissue, such as for example, forceps, clamps, sutures, and bioadhesives. Engaging means 42′ as shown in FIG. 22 may include at least one hooked member at the distal end of engaging means 42′ for engaging tissue surrounding aperture 4′.

Preferably, cable 41 is a flexible coated wire or any other suitable delivery means for delivering engaging means 42′ to an aperture site. Closure device 60 as disclosed may be defined as including the elements of retractor mechanism 40′.

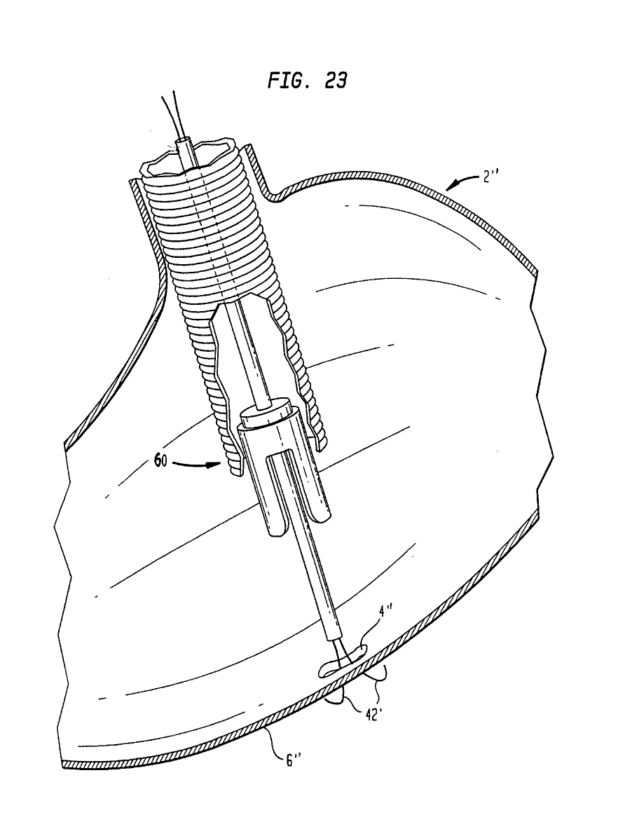

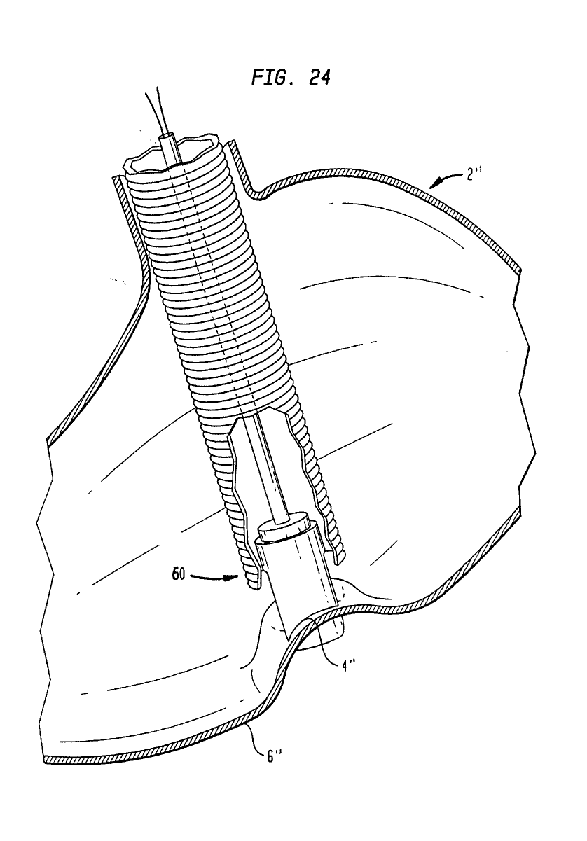

As shown in FIG. 21, the method may further include delivering the retractor mechanism 40″ through aperture 4″ in the lining of the stomach or another organ or portion of the body, and then deploying engaging means 42′ of retractor mechanism 40″ as shown in FIG. 22. Retractor mechanism 40″ may then be pulled back through aperture 4″ as shown generally in FIG. 23 so that engaging means 42′ may engage the tissue surrounding aperture 4″ and pull the tissue between biased legs 144 of clip 140 as seen in FIG. 24. Retractor mechanism 40″ may then be pulled back through endoscope 50′ by pulling on cable 41 connected to the base of trigger 120.

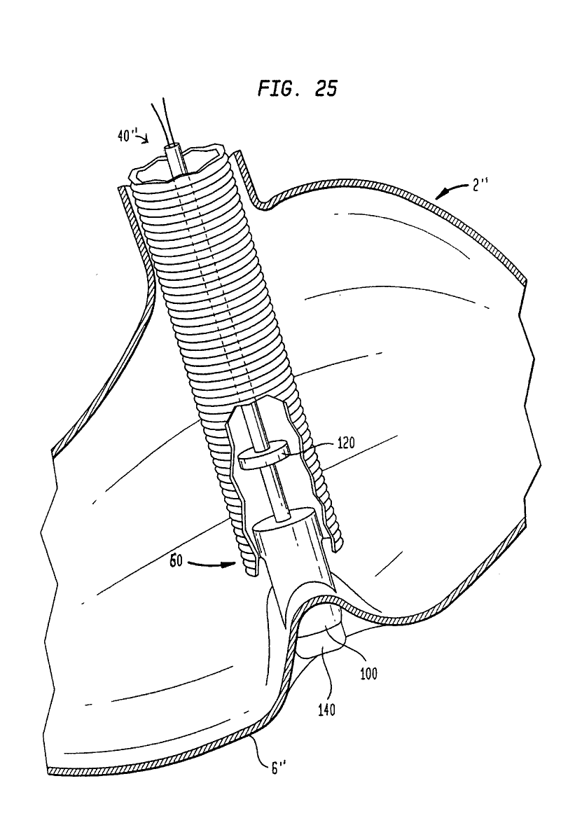

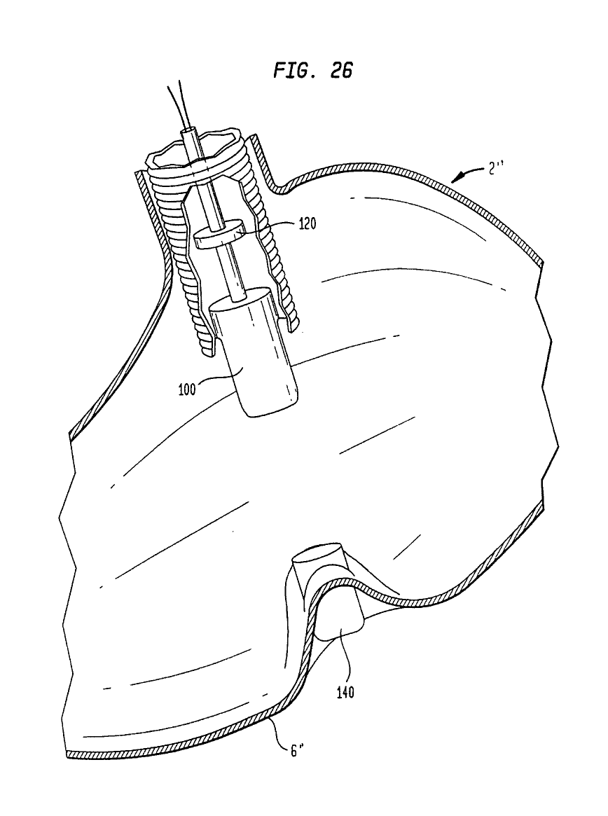

As shown in FIG. 25, retractor mechanism 40″ continues to be pulled away from sealed tissue 6″. As this occurs, trigger 120 of closure device 60 is pulled further away from distal end 105 of housing 100 thereby releasing clip 140 onto the pulled back tissue. After clip 140 is released from trigger 120, the retractor mechanism 40″, trigger 120 and housing 100 may all be removed along with endoscope 50′ from the aperture site as seen in FIG. 26.

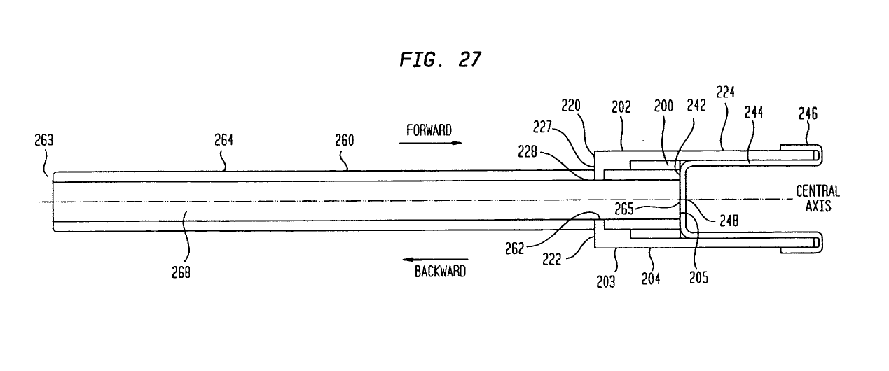

FIGS. 27-35 show an alternative embodiment of an apparatus and method for closing an aperture in the lining of the stomach or another organ or portion of the body. The apparatus may include a central shaft 260 having a proximal end 263, a distal end 265, a central axis generally depicted by a longitudinal dashed line in FIG. 27, and an opening 268 coaxial with the central axis. An outside perimeter 264 of central shaft 260 is preferably configured to fit and move transversely in a forward and backward direction through openings 228, 208, 248 of trigger 220, housing 200, and clip 240. Central shaft 260 is preferably configured to contain hardware used for grabbing tissue and pulling the tissue into clip 240 loaded on legs 224 of trigger 220. Non-exhaustive examples of hardware are a retractor, a bioadhesive, and a balloon as described in previous embodiments. It is within the scope of the invention to include other hardware known in the art such as for example hooks, clamps, and clips.

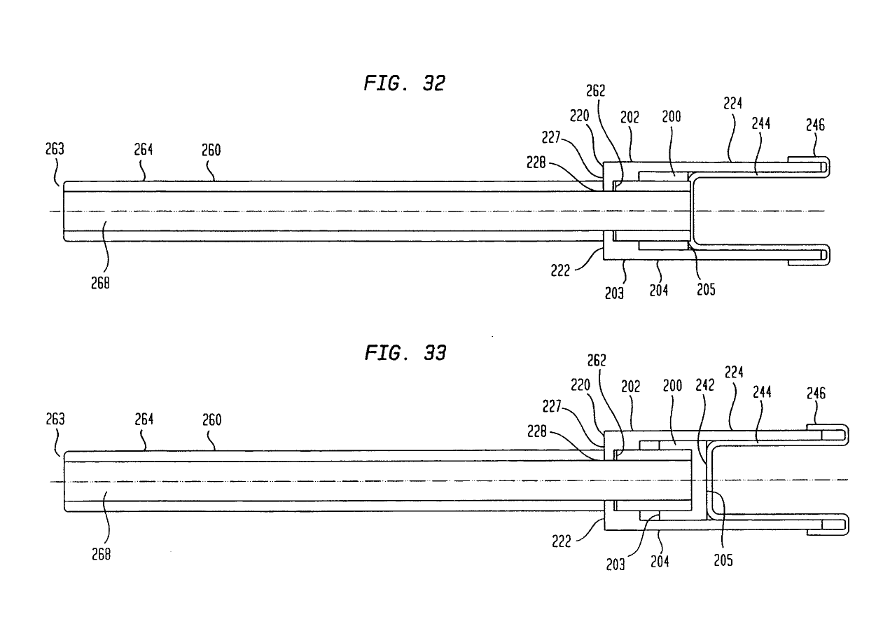

In another aspect of the invention, central shaft 260 may include an engaging means 262. Engaging means 262 may be, for example, a notch in outside surface 264 of central shaft 260. Engaging means 262 may alternatively be, for example, a protrusion on outside surface 264 of central shaft 260. The non-exhaustive examples of a notch and protrusion will preferably be configured to engage a corresponding structure of trigger 220 as shown in FIGS. 32-35. For example, central shaft 260 may have a male feature and the trigger may have a female feature that mate.

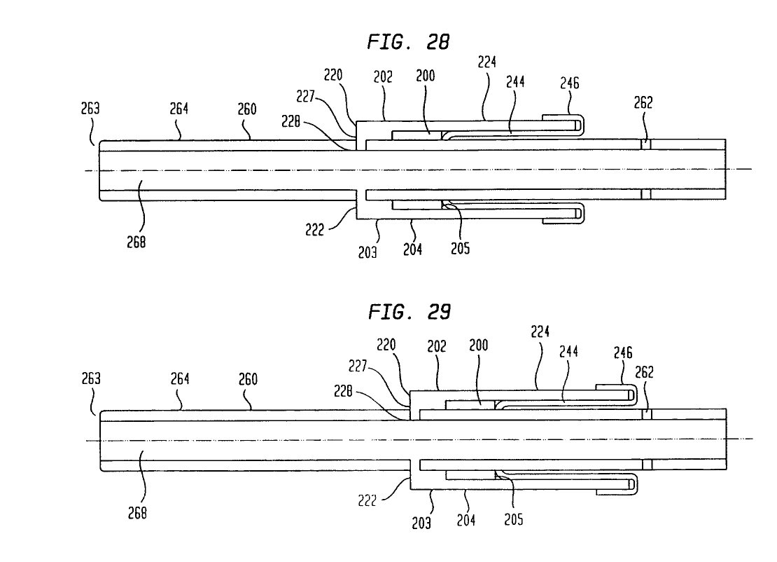

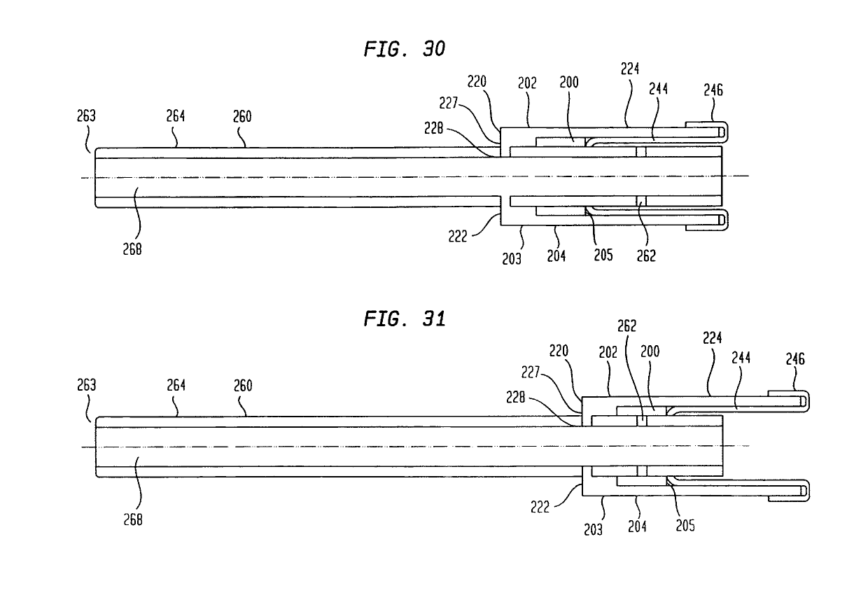

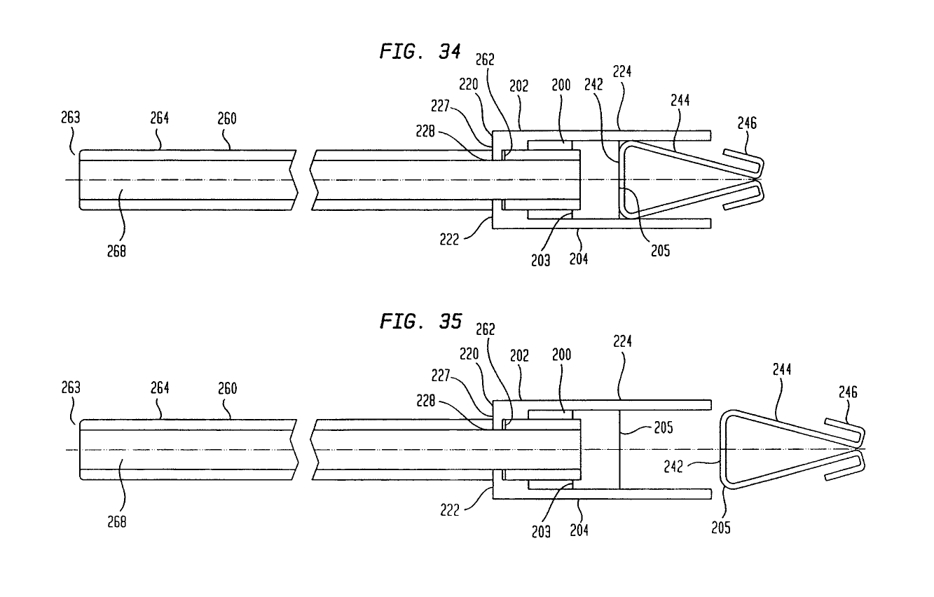

After central shaft 260 has been delivered to an aperture site as shown generally in FIG. 28 through an endoscope for example, and the hardware has been deployed to pull tissue between the loaded clip 240, central shaft 260 is then pulled in the opposite direction back through the endoscope as shown generally in FIG. 29. As central shaft 260 is pulled further back into the endoscope as shown in FIGS. 30-32, engaging means 262 of central shaft 260 eventually engages a mating feature 227 on trigger 220 thereby pulling trigger 220 in the same direction as that of central shaft 260 as shown in FIGS. 33-35. Biased legs 244 of clip 240 are preferably released from legs 224 of trigger 220 as base 222 of trigger 220 is moved further away from distal end 205 of housing 200 while base 242 of clip 240 is prevented from moving to proximal end 203 of housing 200 as shown in FIGS. 34-35. Biased legs 244 may thereby move together and seal tissue therebetween.

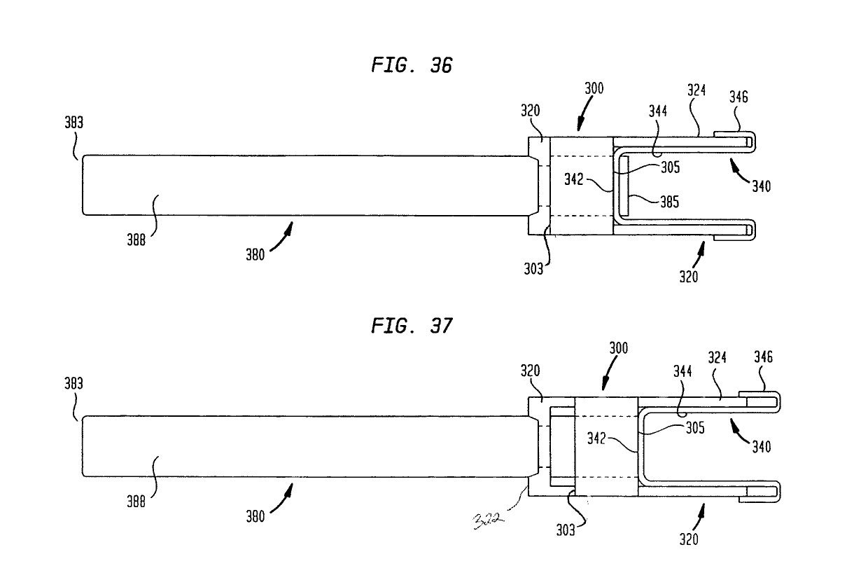

FIGS. 36-39 show an alternative embodiment of an apparatus and method for closing an aperture in the lining of the stomach or another organ or portion of the body. In this embodiment, the apparatus preferably includes an endoscope 380 that engages a distal end 305 of housing 300 and cannot pass therethrough to a proximal end 303 of housing 300. Endoscope 380 may be provided to act as a passageway for surgical instruments and hardware to pass therethrough.

FIGS. 36-39 include endoscope 380, a housing 300, a trigger 320, and a deformable clip 340. A distal end 385 of endoscope 380 cannot pass through housing 300 from distal end 305 of housing 300 to the proximal end 303. Endoscope 380 and trigger 320 are generally attached. As seen in FIG. 37, base 322 of trigger 320 has been pulled further away from distal end 305 of housing 300 by movement of endoscope 380 in the same direction. Hooked ends 346 of clip 340 have begun to separate from legs 324 of trigger 320.

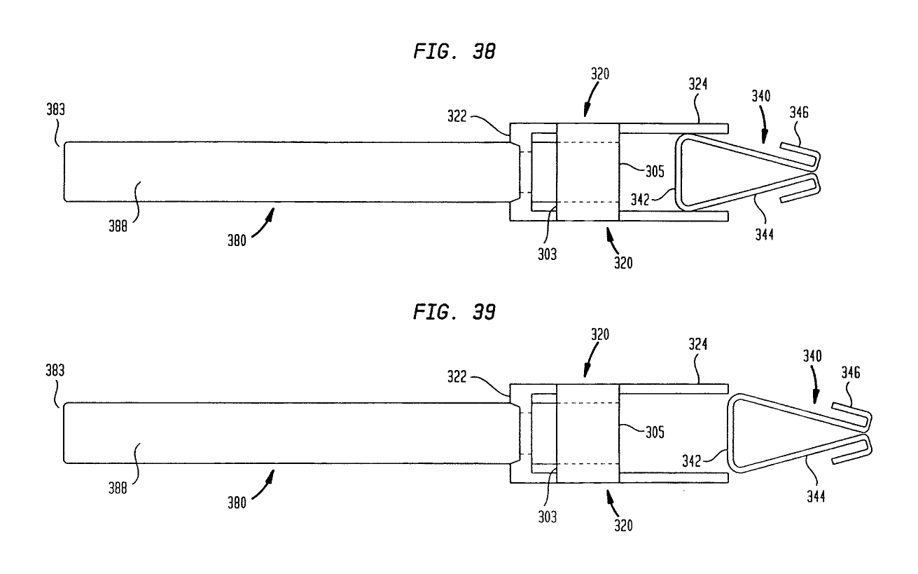

FIGS. 38-39 depict clip 340 being released from trigger 320 and endoscope 380, trigger 320, and housing 300 being removed from the sealed tissue site. In FIG. 38, clip 340 has been released from trigger 320. Biased legs 344 of clip 340 can be seen biased together. Not shown is tissue surrounding the aperture site sealed by biased legs 344 of clip 340.

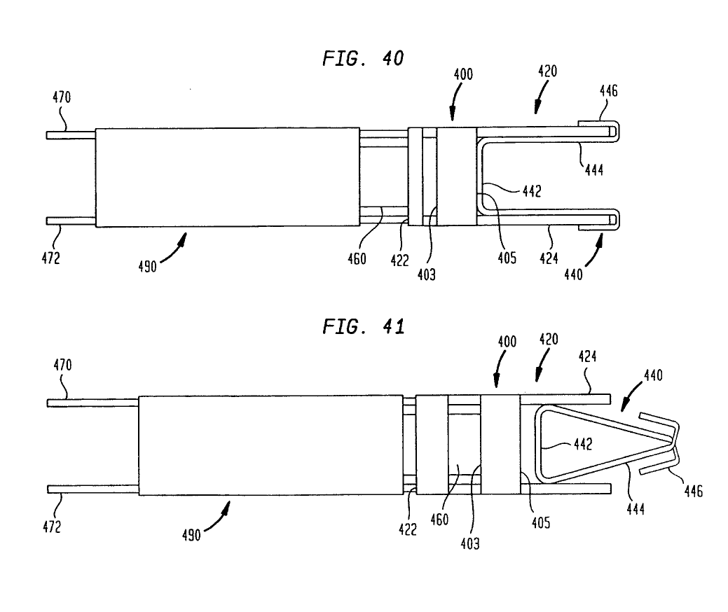

FIGS. 40-41 show an alternative embodiment of an apparatus and method for closing an aperture in the lining of the stomach or another organ or portion of the body. In this embodiment, the apparatus may include an overtube 490 for containing a first bar 470 and a second bar 472. The first and second bars 470, 472 are preferably connected to base 422 of trigger 420 for pulling trigger 420 away from distal end 405 of housing 400 and releasing clip 440. First and second bars 470, 472 may be attached to housing 400 and provide a track means for trigger 420 slidably connected to housing 400 to move further away from distal end 405 of housing 400 as endoscope 460 engages trigger 420. Overtube 490 may be attached to and separated from housing 400 a fixed distance. Overtube 490 is preferably configured to guide trigger 420, housing 400, clip 440, and endoscope 460 to the aperture site in tissue. Overtube 490 may be configured to provide a passageway for hardware delivered to the aperture site.

In another aspect of the invention, the first and second bars 470, 472 may be, for example, wires, cables, or rods. First and second bars 470, 472 may be attached to the trigger. First and second bars 470, 472 may extend out of overtube 490 and may be accessible for a surgeon to pull on outside of the overtube 490. As first and second bars 470, 472 are pulled, trigger 420 is preferably pulled further away from distal end 405 of housing 400 and biased legs 444 of clip 440 are released from legs 424 of trigger 420. Biased legs 444 may thereby move together and seal tissue therebetween.

In an alternative embodiment, an additional endoscope, not shown in FIGS. 40-41, may contain overtube 490, first and second bars 470, 472, endoscope 460, trigger 420, housing 400, and clip 440. The additional endoscope may be positioned around an aperture in the lining of the stomach or another organ or portion of the body. Preferably, a distal end of the additional endoscope, the end that surrounds the aperture, is c-shaped. This shape preferably allows more tissue to be placed between biased legs 444 of clip 440 in the open position before clip 440 is released to seal the tissue at the aperture site.

In a non-limiting method of this embodiment, overtube 490, first and second bars 470, 472, endoscope 460, trigger 420, housing 400, and clip 440 may all be contained within the additional endoscope as it is positioned around the aperture. Preferably, the additional endoscope is held over the aperture site while surgical instruments, hardware and/or a closure device are delivered though the endoscope to the aperture site. A combination of a retractor mechanism, a bioadhesive, a bioabsorbable, or a bioresorbable material, and/or balloon may be delivered into the aperture through the lining of the stomach or another organ or portion of the body and then used to retract at least a portion of tissue into the endoscope containing the closure mechanism. The retracted tissue preferably is pulled between biased legs 444 of clip 440 of the closure mechanism when clip 440 is in the open position. Trigger 420 holds clip 440 in the open or loaded position.

Preferably, when trigger 420 is moved further away from distal end 405 of housing 400 by first and second bars 470, 472 attached to trigger 420, trigger 420 releases clip 440. Preferably, clip 440, when released by trigger 420, closes down on the retracted tissue to seal the gastrotomy. After clip 440 has been released, all instruments may be removed from the aperture site. Preferably, the additional endoscope containing overtube 490, first and second bars 470, 472, endoscope 460, trigger 420, housing 400, and clip 440 may all be removed.

Although the invention herein has been described with reference to particular embodiments, it is to be understood that these embodiments are merely illustrative of the principles and applications of the present invention.

1. An apparatus for sealing an aperture in body tissue that extends from an outside surface to an inside surface of the body tissue, comprising:
an expandable member adapted to be at least partially inserted into said aperture; and
an expandable material positioned against at least a portion of said expandable member, said expandable material having a sealing surface adapted to be at least inserted through the aperture in an unexpanded state and to expand against an inside surface of said body tissue surrounding said aperture,
wherein said expandable member is adapted to expand said expandable material such that said sealing surface of said expandable material comes in contact with the inside surface of the body tissue to seal said aperture.

2. The apparatus of claim 1, further including an endoscope, said endoscope having a cannula through which said expandable member and said expandable material may pass to enter through said aperture in said body tissue.

3. The apparatus of claim 1, wherein said expandable member is an inflatable balloon and said expandable material is a bioadhesive.

4. The apparatus of claim 1, wherein said expandable material further comprises a therapeutic agent.

5. The apparatus of claim 1, wherein said expandable material includes an opening adapted to allow said expandable member to exit therefrom.

6. The apparatus of claim 5, wherein said expandable material further includes at least one retaining mechanism adapted to be engaged by a retractor for sealing said opening in said expandable material.

7. An apparatus for sealing stomach tissue surrounding an aperture in the lining of a stomach that extends from an outside surface of the stomach to an inside surface of the stomach, comprising:
a first expandable material adapted to be at least partially inserted into said aperture; and
a second expandable material positioned against at least a portion of said first expandable material, said second expandable material having a bioadhesive sealing surface adapted to be at least inserted through said aperture in an unexpanded state and to expand along said inside surface of said stomach tissue surrounding said aperture as said first expandable material is expanded by said second expandable material.

8. A method of sealing an aperture in body tissue that extends from an outside surface to an inside surface of the body tissue, comprising:
delivering an expandable material positioned against at least a portion of an expandable member into said aperture, said expandable material having a sealing surface adapted to be at least inserted through said aperture in an unexpanded state; and
expanding said expandable member such that said expandable material comes in contact with the inside surface of said body tissue to seal said aperture.

9. The method of claim 8, further comprising:
placing an endoscope on said outside surface of said body tissue surrounding said aperture, said endoscope having a cannula through which said expandable member and said expandable material may pass; and
delivering said expandable member and said expandable material at least partially through said aperture.

10. An apparatus for sealing an aperture in body tissue that extends from an outside surface to an inside surface of the body tissue, comprising:
a trigger including a base having two legs extending substantially perpendicular therefrom;
a clip including a base having two biased legs extending therefrom, the two biased legs of said clip releasably engaged to said two legs of said trigger; and
a retractor adapted to engage and pull said body tissue surrounding said aperture between said two biased legs of said clip, such that when said biased legs of said clip are released from said legs of said trigger, said biased legs seal said body tissue surrounding said aperture therebetween.

11. The apparatus of claim 10, further comprising:
a housing having a base configured to engage said base of said clip, wherein said base of the said housing inhibits said base of said clip from moving in the same direction as that of said trigger as said clip is released from said trigger.

12. The apparatus of claim 10, wherein each base of said trigger and clip include an opening for receiving said retractor therethrough.

13. The apparatus of claim 12, wherein said openings of said trigger and clip are coaxial.

14. An apparatus for sealing an aperture in body tissue that extends from an outside surface to an inside surface of the body tissue, comprising:
a trigger including a base having two legs extending substantially perpendicular therefrom;
a clip including a base having two biased legs extending therefrom, the two biased legs of said clip releasably engaged to said two legs of said trigger;
a central shaft configured to engage the base of said trigger; and
a retractor having means to engage and pull said body tissue surrounding said aperture between said two biased legs of said clip, such that when said central shaft engages said trigger and moves said trigger in a backward direction, said biased legs of said clip are released from said legs of said trigger sealing said body tissue surrounding said aperture therebetween.

15. The apparatus of claim 14, further comprising:
a housing having a base configured to engage said base of said clip, wherein said base of the said housing inhibits said base of said clip from moving in the same direction as that of said trigger as said clip is released from said trigger.

16. The apparatus of claim 14, wherein said trigger, clip, and central shaft each include an opening for receiving said retractor therethrough.

17. The apparatus of claim 16, wherein said openings of said trigger, clip, and central shaft are coaxial.

18. The apparatus of claim 14, wherein said central shaft includes an engagement portion for engaging said base of said trigger.

19. A method of sealing an aperture in body tissue that extends from an outer surface to an inner surface of the body tissue, comprising:
pulling said body tissue surrounding said aperture with a retractor between two biased legs of a clip in an open first position, said biased legs releasably engaged to two legs of a trigger;
releasing said two biased legs of said clip from said legs of said trigger into a closed second position sealing said body tissue surrounding said aperture therebetween.

20. The method of claim 19, further comprising:
placing an endoscope on an outside surface of said body tissue surrounding said aperture; and
delivering said retractor through said aperture.
